(19) United States
(12) Patent Application Publication
(10) Pub. No.: US 2016/0102066 A1
(43) Pub. Date: Apr. 14, 2016

(54) BENZOTHIAZOLE DERIVATIVE AND ANTI-TUMOR USE THEREOF

(71) Applicant: YANTAI UNIVERSITY, Yantai (CN)

(72) Inventors: Hongbo WANG, Yantai (CN); Xuechuan HONG, Wuhan (CN); Xi ZHU, Wuhan (CN); Pengyu WANG, Wuhan (CN); Guangyao LV, Yantai (CN); Jie FU, Wuhan (CN); Huairong LUO, Wuhan (CN); Jianqiao ZHANG, Yantai (CN); Meng WEN, Wuhan (CN); Chunrong QU, Wuhan (CN); Jinmei ZHU, Wuhan (CN); Xianming HU, Wuhan (CN)

(73) Assignee: YANTAI UNIVERSITY, Yantai (CN)

(21) Appl. No.: 14/719,765

(22) Filed: May 22, 2015

(30) Foreign Application Priority Data
Oct. 9, 2014 (CN) 201410527249.8

(51) Int. Cl. C07D 277/82

(52) U.S. Cl. CPC C07D 277/82

(57) ABSTRACT

The present invention relates to a benzothiazole derivative of formula 1 or a pharmaceutically acceptable salt thereof and a process for preparation thereof. The present invention also relates to a pharmaceutical composition comprising the compounds and the use of the compounds in the preparation of an anti-tumor medicament.

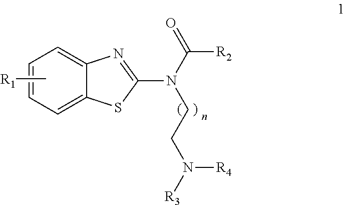

### TECHNICAL FIELD

The present invention belongs to the field of organic synthesis, and relates to an anti-tumor drug benzothiazole derivative and use thereof.

### BACKGROUND

With the extension of human life, cancer emerged as the leading cause of death in recent years. “2012 Chinese Cancer Registry Annual Report” shows that about 3.5 million of new cancer cases occur and about 2.5 million of persons die of cancer each year in China. Lung cancer has a highest incidence among malignant tumors in China, followed by stomach cancer, colorectal cancer, liver cancer and esophageal cancer. Cancer has become the leading cause of human death in China.

With regard to treatment of cancers, scientists have carried out a tot of research work. New anticancer drugs are discovered continuously Currently, there are more than 20 kinds of cancers, cure rates of which are above 30%. The research of drug action mechanism at sub-cellular and molecular level largely expands the research in the application of anti-cancer drugs. The rapid development of cell kinetics, pharmacokinetics and immunological research makes the drug screening, dose titeration, and determination of route of administration become more and more mature. Now treatments of malignant tumors have achieved very good therapeutic effects by means of combination therapy, high-dose intermittent therapy, adjuvant chemotherapy, and therapy in combination with traditional Chinese medicine. Nowadays the means for treatment of cancer are mainly surgery therapy, radiation therapy, chemotherapy, traditional Chinese medicine therapy and immunotherapy etc. The choice of anti-cancer drugs, toxicity and drug resistance etc. affect the efficacy since the anti-cancer drugs can kill not only tumor cells, but also cells of normal tissues, especially quickly proliferative hematopoietic cell in bone marrow and stomach intestinal cells. This limits doses of anti-cancer drugs, and reduces immune function in patients. Worse still, it can result in the failure of the treatment as a result of the unbearable gastrointestinal reactions which force the patients to discontinue the treatment. Anti-cancer drugs can kill cancer cells, but also have cytotoxicity. So, it is always a goal of scientists to find a drug which can treat cancer and have no or little harm to human. Recently, research on the relationship between TRPC6 protein as one member of subfamily of transient receptor potential channels, the change of intracellular calcium concentration, development of tumor, and changes of tumor cell cycle has made new progress. TRPC6 is expected to become a new target for cancer therapy.

Transient receptor potential channel (TRPC) is a non-selective cation channel protein family commonly found in the cell membrane, and plays an important role in mediating sensory conduction, cell signal transduction and regulation of development etc. Currently, it is one of hotspots in research field of ion channels. TRP channel proteins are a large family, and are widely expressed in a variety of organisms, tissues and cells. As far as mammalian TRP channels are concerned, this family includes seven interrelated sub-families: TRPC, TRPV, TRPM, TRPN, TRPA, TRPP and TRPML, each of which in turn comprises a number of family members. The previous research on TRP ion channels was restricted to the nervous system. Recent studies have shown that TRP channels play an important role not only in cellular signal transduction, mediating nociception etc. in the body, but also in tumor occurrence and development. The family has a stabilizing and regulating effect on cells, its increased expression promotes growth of malignant tumors.

TRPCs, namely the traditional TRP channel, are the first TRP channel proteins which are isolated and researched. TRPC has 7 subtypes, namely TRPC (1˜7), wherein TRPC3 and TRPC6 are very similar in structure and function, and the identity of amino acids is as high as 70%-80%. Besides, their pharmacological properties and signal regulating functions are also similar. They are more representative in TRPC subfamily, and are two subtypes concerned in the current international research. And TRPC6 is considered as the most selective channel protein. Human TRPC6 locates on chromosome 11q212q22, has a total of 132,287 bases (gene pool: NC000011), and contains 13 exons. The mRNA as transcription product of TRPC6 contains 4,564 bases, wherein the 1-427 positions are 5′ untranslated region, the 428-3,223 positions are coding region, the 3,224-4,564 positions are 3′ untranslated region (gene pool: NM004621). TRPC6 can be specifically activated by phospholipase C (PLC), subsequently lead the ligand to bind to the membrane receptor by G-protein coupled receptor (GPCR) mediated signal transduction pathway, then activate phospholipase C to generate 1,4,5-inositol triphosphate, which binds to a receptor to promote the Ca²⁺ release from the endoplasmic reticulum. TRPC6 is a non-selective cation channel through which calcium ions can pass, and is expressed in many tissues. It can directly be activated by the second messenger diacylglycerol enzymes, subsequently intracellular calcium flux is changed by phosphorylation regulation of particular tyrosine/serine. The increase of intracellular free Ca²⁺ activates some protein phosphatases, resulting in the phosphorylation of substrate proteins. The external signals are enlarged by cascade amplification, then enter the nucleus and affect the DNA replication, leading to malignant transformation of cells as well as proliferation and differentiation of tumor cells. Intracellular Ca²⁺ directly involves in the regulation of growth, invasion, metastasis, and differentiation of tumors. Therefore, TRPC6 inhibitors are expected to become new drugs to treat cancer. However, there are few reports about TRPC6 inhibitors.

In recent years, scientists have conducted a series of studies on the relationship between TRPC6 and human tumors. The results demonstrate that TRPC6 is closely associated with the higher incidence of gastric cancer, liver cancer, esophageal cancer and so on. David G. W. reported the TRPC3 and TRPC6 inhibitors in 2013. The compounds synthetized by them have IC₅₀ values that can reach nanomolar order for hTRPC3 and hTRPC6. However, as for animal experiments, the series of drugs were found to have low oral bioavailability and unduly high in vivo clearance rate. Even after a series of structural modification, the people still cannot find a balance point to make both the activity and oral bioavailability arrive at a good level.

Through a large number of screening, we found that compound 1 has excellent TRPC6 inhibitory effect and is a potential antitumor drug.

### CONTENTS OF THE INVENTION

The technical problem to be solved by the present invention is to provide a benzothiazole derivative. The present invention also provides activity screening results of said compound at cellular level and target level and the antitumor use thereof.

The present invention relates to a benzothiazole derivative, having the structure as shown in formula 1:

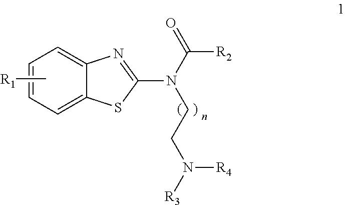

wherein:
R₁ is hydrogen, alkyl, alkoxy, alkylcarbonyl or alkoxycarbonyl;
R₂ is phenyl and substituted phenyl, pyridine, imidazole, furan and substituted heterocycle, the substituents include halo-substituted alkyl and halogen;
R₃ and R₄ are independently hydrogen, alkyl or aryl;
n is an integer from 1 to 6;
said alkyl is C₁-C₆ linear, branched or cyclic alkyl group such as methyl, ethyl, n-propyl, isopropyl, n-butyl, cyclohexyl and the like;
said haloalkyl is a haloalkyl substituted by 1-5 halogen atoms, such as a monochloromethyl, trifluoroethyl and the like;
said alkoxycarbonyl is a C₁-C₆ alkoxycarbonyl, such as methoxycarbonyl, ethoxycarbonyl, t-butoxycarbonyl group and the like;
said alkylcarbonyl is a C₁-C₆ alkylcarbonyl group, such as formyl, acetyl, propionyl, isobutyryl and the like;
said halogen is fluorine, chlorine, or bromine.

The present invention provides a compound of formula I or a pharmaceutically acceptable salt thereof.

The term “pharmaceutically acceptable salt” as used in the present invention refers to, within the range of reliable medical evaluation, the salt of the compound is suitable to contact tissues of a human or lower animals without undue toxicity, irritation and allergy etc., has a reasonable benefit/risk ratio, is generally soluble or dispersible in water or oil, and is effective for their intended use. The salts which can be used herein and are compatible with the chemical property of the compound of formula 1 include pharmaceutically acceptable acid addition salts and pharmaceutically acceptable base addition salts.

The present invention also provides a process for the preparation of benzothiazole derivatives as shown by formula 1.

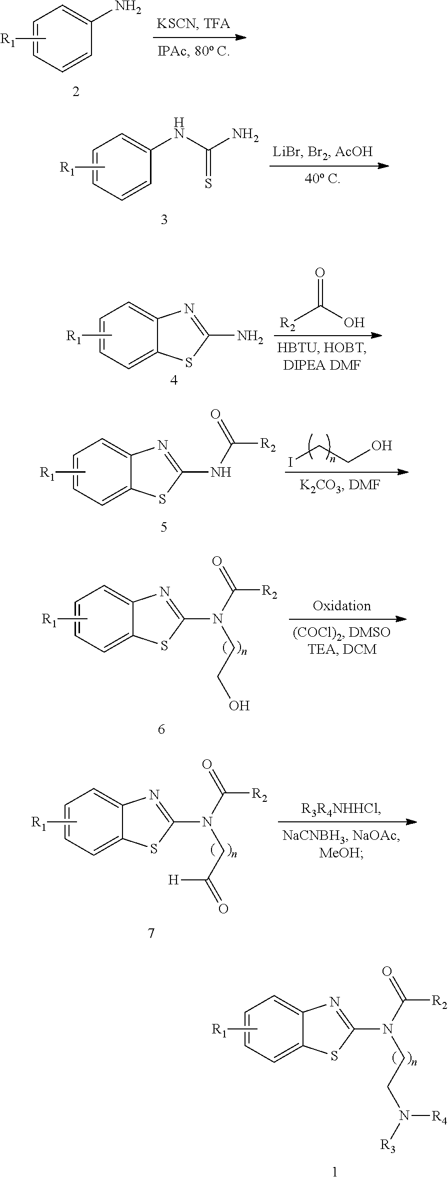

### DESCRIPTION OF FIGURES

FIG. 1 is ¹H spectrum of compound 1-1 of example 1 in (CD₃)₂SO.

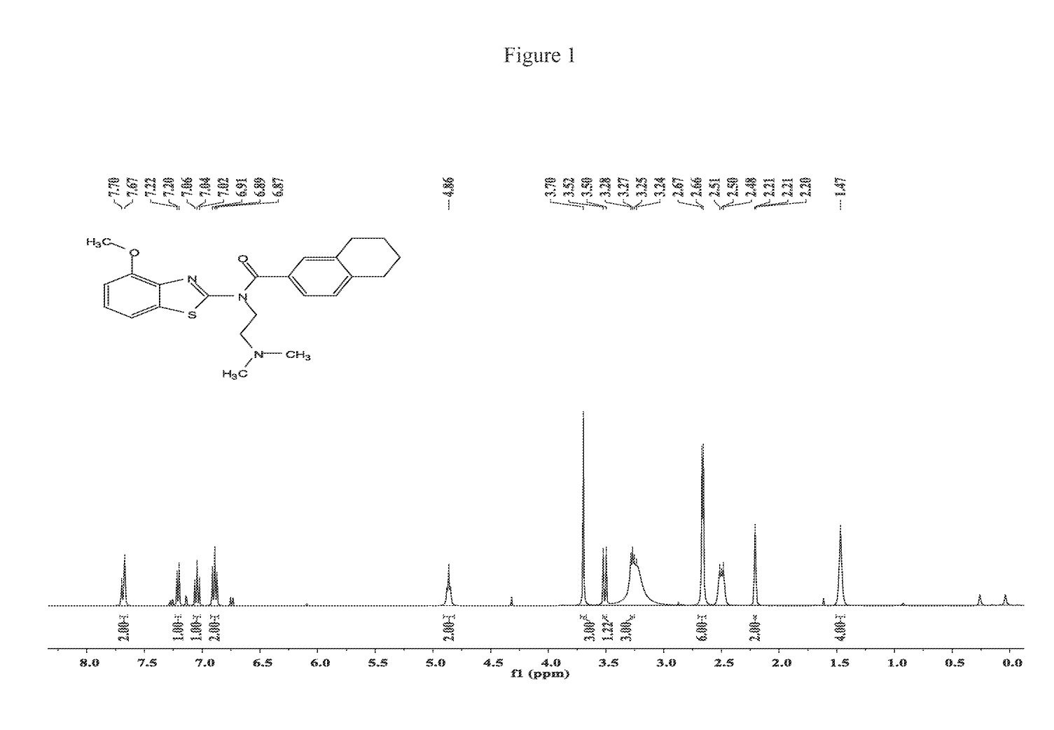

FIG. 2 is ¹³C spectrum of compound 1-1 of example 1(CD₃)₂SO.

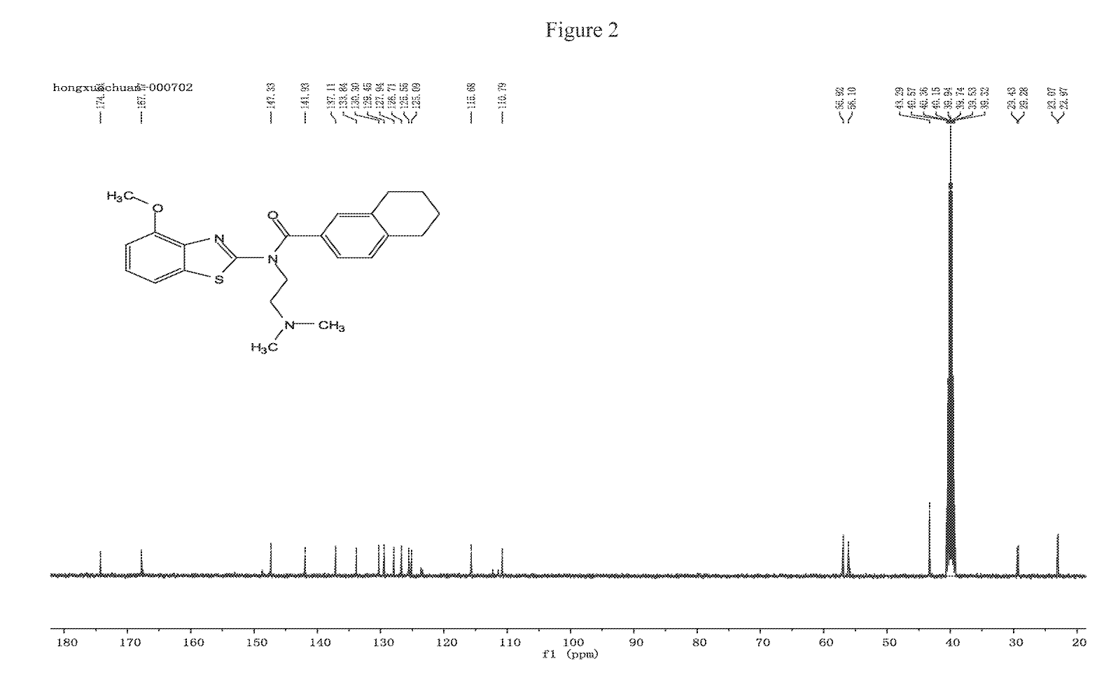

### EXAMPLES

The following examples are helpful to understand the present invention, but they cannot be explained as limiting the scope of the present invention.

### Example 1

R₁=—OCH₃,

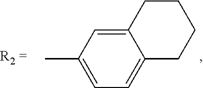

R₃=R₄=—CH₃, n=1.

Compound 2 (3.70 g, 0.03 mol) and potassium thiocyanate (4.38 g, 0.045 mol) were dissolved in butyl acetate (30 ml). In an ice bath, TFA (5.74 ml, 0.075 mol) was slowly added dropwise. The mixture was warmed to 80° C. and stirred for 17 h, cooled to room temperature. 6 ml pure water was added, cooled to 0° C. and kept for 1 h. The mixture was filtered by suction under reduced pressure, and then washed with water (grade II), and dried in a vacuum oven to give a white solid 3 (4.77 g, 84.57%).

(1) Compound 3 (3.76 g, 0.02 mol) was dissolved in acetic acid (36 ml). To which was added lithium bromide (2.6 g, 0.03 mol) at room temperature, and bromine (1 ml, 0.02 mol) was slowly added dropwise in an ice bath, and then heated to 40° C., stirred for 17 h. The reaction system was lowered to room temperature and kept for 2 h. The reaction solution was filtered by suction and washed with acetic acid, and dried in a vacuum oven to give a white solid 4 (3.5 g, 94.08%).

(2) Compound 4 (1.86 g, 0.01 mol) was placed in a round bottom flask. Then 5,6,7,8-tetrahydroxy-2-naphthoic acid (1.85 g, 0.0105 mol), HOBT (4.05 g, 0.03 mol) and HBTU (11.38 g, 0.03 mol) were added and dissolved with DMF (60 ml). Then DIPEA (15.67 ml, 0.09 mol) was added dropwise, and stirred at room temperature. After the completion of reaction, the reaction was dissolved with ethyl acetate (250 ml), and then extracted with water (3×60 ml) to remove DMF, and washed with saturated brine, dried over anhydrous magnesium sulfate, filtered. Separation by silica gel column (ethyl acetate: petroleum ether=1:8) provides compound 5 (2.05 g, 60%).

(3) Compound 5 (1.41 g, 0.0043 mol) and potassium carbonate (1.78 g, 0.0129 mol) were added into a round bottom flask. The flask was made free of water and oxygen. The mixture was dissolved with DMF (60 ml). Then 2-iodo-ethanol (0.67 ml, 0.0086 mol) was slowly added dropwise. The reaction was raised to 50° C. and stirred. After completion of the reaction, the reaction was dissolved with ethyl acetate (250 ml), and then extracted with water (3×60 ml) to remove DMF, and washed with saturated brine, dried with anhydrous magnesium sulfate, filtered, evaporated to dryness, and dried by pumping to provide a white solid 6 (1.475 g, 93.35%).

(4) Oxalyl chloride (0.32 ml, 0.00377 mol) was dissolved in dichloromethane (40 ml), and cooled to −78° C. and vigorously stirred. Then DMSO (0.54 ml, 0.00754 mol) was added dropwise. And then the compound 6 (1.107 g, 0.0029 mol) was dissolved in dichloromethane (15 ml). After 15 min, the solution of compound 6 in dichloromethane was slowly added dropwise to the solution of oxalyl chloride in dichloromethane, 15 min later, to the reaction solution was added dropwise triethanolamine (2.02 ml, 0.0145 mol), then moved to room temperature and kept for 45 min. After completion of the reaction, the reaction was quenched with saturated ammonium chloride solution (50 ml) and extracted with dichloromethane (3×40 ml), washed with saturated brine, dried with anhydrous magnesium sulfate, filtered, evaporated to dryness, and dried by pumping to provide a white solid 7 (1.03 g, 93.50%).

(5) Compound 7 (1.1 g, 0.0029 mol) was dissolved in methanol (150 ml). Then to which was added dimethylamine hydrochloride (1.182 g, 0.0145 mol) in an ice bath, stirred at room temperature for 0.5 h. Then 4 Å molecular sieves (2.064 g) was added, stirred at room temperature for 8 h, and then sodium cyanoborohydride (0.365 g, 0.0058 mol) was added in an ice bath. The mixture was stirred overnight. After completion of the reaction, methanol was evaporated to dryness. The residue was dissolved with water, extracted with ethyl acetate, washed with saturated brine, dried with anhydrous magnesium sulfate, filtered, evaporated to dryness, and dried by pumping to give compound 14 (0.82 g, 69%). 1H NMR (400 MHz, DMSO) δ 7.68 (d, J=10.3 Hz, 2H), 7.21 (d, J=7.8 Hz, 1H), 7.04 (t, J=8.0 Hz, 1H), 6.89 (t, J=8.1 Hz, 2H), 4.86 (s, 2H), 3.70 (s, 3H), 3.51 (d, J=10.7 Hz, 1H), 3.28 (d, J=5.5 Hz, 3H), 2.66 (d, J=4.6 Hz, 6H), 2.22-2.20 (m, 2H), 1.47 (s, 4H).

### Example 2

### The Cytotoxic Activity Screening Test of the Compound of the Present Invention at Cellular Level

After the cells in logarithmic growth phase were digested with 0.25% trypsin −EDTA, the cell suspension at a certain concentration was prepared. Depending on cell growth rate, they were inoculated in 96-well plates at 1000-2000 cells/well. To each well 100 μL of the cell suspension was added. After 24 h, fresh media containing various concentrations of the compounds or the corresponding solvent were added at 100 μL per well (DMSO final concentration <0.1%). 5 to 7 dose groups were arranged for each test compound. Each group comprised at least 3 parallel wells. After further culturing at 37° C. for 72 h, the supernatant was discarded. 100 μL of fresh scrum-free medium containing 0.5 mg/mL MTT was added to each well, further cultured for 2 h. After the supernatant was discarded, to each well was added 200 μL DMSO to dissolve MTT formazan precipitate. After mixing homogeneously by a microoscillator, optical density (OD) was measured at 450 nm of reference wavelength, and at 570 nm of detection wavelength by a Microplate reader. The tumor cells treated by the solvent control was used as the control group. The inhibition rate of tumor cells for the compounds were calculated by the following equation and IC₅₀ was calculated by the half of inhibition equation:

$$Inhibition rate ( \% ) = \frac{\begin{matrix}the average OD value of the control group - \\ the average OD value of the treated group\end{matrix}}{the average OD value of the control group} * 100 \%$$

(The results are shown in Table 1)

TABLE 1
The results of cytotoxic activity in vitro
of compound of the present invention

| | IC₅₀ (μM) |
|---|---|
| Cell lines | 1-1 |
| MCF7 | 2.53 |
| H460 | 27.24 |
| KB | 4.59 |
| Lncap | 8.41 |
| Pc3 | >100 |
| Du145 | 12.89 |
| RM-1 | 33.91 |

MCF7 is human breast cancer cell line; H460 is human lung cancer cell line; Lncap, Pc3, Du145 is human prostate cancer cell lines; KB is human oral epithelium carcinoma cell line; RM-1 is mouse prostate cancer cell line.

### Example 3

### Animal Activity Experiments In Vivo of Compounds of the Invention

Male C57/BL6 mice (18-22 g) were used. The experiment procedures are summarized as follows: mice who had prostate cancer RM-1 and grown well were sacrificed by cervical dislocation. Under sterile conditions, tumor mass growing well was stripped off, homogenized, diluted with physiological saline in 1:4. Each mouse was inoculated 0.2 mL of the tumor solution in the armpit and back (approximately 2*10⁶ cells). On the next day; the animals were randomized into groups and the administration began. Compound 1-1 was administered at 50 mg/10 g by intraperitoneal injection at 24 h after inoculation. The dosing volume was 0.1 mL/10 g. At the same time, docetaxel control group and solvent control group were set. As for the docetaxel group, the animals were administered by intraperitoneal injection at 10 mg/kg dose, the dosing volume was 0.1 mL/10 g. After continuous administration for 10 days, the animals were sacrificed by cervical dislocation, weighed the body and the tumor, respectively. Inhibition rate of tumor growth (%) was calculated, and the results were statistically processed.

$$Inhibition rate of tumor ( \% ) = \frac{\begin{matrix}average tumor weight of control group - \\ average tumor weight of treatment group\end{matrix}}{average tumor weight of control group} * 100 \%$$

TABLE 2
The effect of the compound of the present invention
on mice prostate cancer RM-1 transplanted tumor
weight and body weight of the animals

| | dose | body weight of | | | Inhibi- |
|---|---|---|---|---|---|
| | (mg/ | Animals (g) | | Tumor | tion |
| Group | kg) × d | Initial | Final | weight (g) | rate |
| Control | | 17.92 ± 1.07 | 21.50 ± 1.41 | 2.59 ± 0.47 | |
| Docetaxel | 10 × 3 | 20.00 ± 1.07 | 18.43 ± 2.17 | 1.77 ± 0.61 | 32% |
| 1-1 | 100 × 4 | 19.38 ± 1.06 | 21.00 ± 0.10 | 1.73 ± 0.11 | 33% |

The above experimental results show that the compound 1-1 having the general formula according to the present invention has a pharmacological activity in vivo to inhibit growth of prostate cancer RM-1 transplanted tumor in mice.

1-7. (canceled)

8. A method for treating a cancer associated with abnormal activity of TRPC6 comprising administering an effective amount of a compound which is 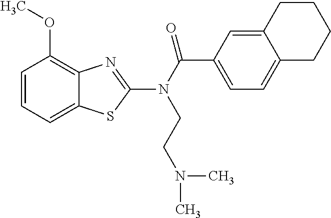 [SMILES: COc1cccc2sc(N(CCN(C)C)C(=O)c3ccc4c(c3)CCCC4)nc12]
or a pharmaceutically acceptable salt or prodrug thereof to a subject in need thereof.

9. The method according to claim 8, wherein the cancer is selected from breast cancer, lung cancer, prostate cancer and oral epithelium carcinoma.
